(19) United States
(12) Patent Application Publication
(10) Pub. No.: US 2022/0265128 A1
(43) Pub. Date: Aug. 25, 2022

(54) MULTIPLE PLANE X-RAY IMAGING CAPSULE

(71) Applicant: CHECK-CAP LTD., Isfiya (IL)

(72) Inventor: Yoav KIMCHY, Haifa (IL)

(21) Appl. No.: 17/627,818

(22) PCT Filed: Oct. 15, 2020
(86) PCT No.: PCT/IL2020/051098

§ 371 Date: Jan. 18, 2022

Related U.S. Application Data
(60) Provisional application No. 62/915,648, filed on Oct. 16, 2019.

(51) Int. Cl. A61B 1/04; A61B 1/273; A61B 6/00

(52) U.S. Cl. CPC A61B 1/041; A61B 1/273; A61B 6/4057; A61B 6/425

(57) ABSTRACT

An imaging capsule, including a radiation source; a collimator that blocks the emission of radiation from the radiation source except through two or more output columns; a detector paired to each output column configured to detect particles resulting from X-ray fluorescence and/or Compton backscattering in response to the particles emitted by the output columns; wherein the collimator is configured to rotate around an X axis to scan a partial or full inner circumference of a user's colon with radiation emitted from each output column; and wherein at least two of the two or more output columns are tilted by a distinct angle relative to a Y axis that is perpendicular to the X axis, to scan distinct positions along the user's colon and form images of a slice of the colon in parallel planes.

### RELATED APPLICATIONS

The present application claims priority from U.S. Provisional application No. 62/915,648 filed on Oct. 16, 2019, the disclosure of which is incorporated herein by reference.

### FIELD OF THE INVENTION

The present invention relates generally to reconstructing an image of a patient's gastrointestinal tract with an imaging capsule using radiation, and more specifically to imaging with radiation in multiple planes.

### BACKGROUND OF THE INVENTION

One method for examining the gastrointestinal tract for the existence of polyps and other clinically relevant features that may indicate regarding the potential of cancer is performed by swallowing an imaging capsule that will travel through the tract and view the patient's situation. In a typical case the trip can take between 24-48 hours after, which the imaging capsule exits in the patient's feces. Typically the patient swallows a contrast agent to enhance the imaging ability of the imaging capsule. Then the patient swallows the imaging capsule to examine the gastrointestinal tract while flowing through the contrast agent. The imaging capsule typically includes a radiation source, for example including a radioisotope that emits X-rays or Gamma rays. The radiation is typically collimated to allow it to be controllably directed toward a specific area during the imaging process. In an exemplary case the imaging capsule is designed to detect particles from X-ray fluorescence and/or Compton back-scattering responsive to the radiation and transmit measurements (e.g. a count rate) to an external analysis device, for example a computer or other dedicated instruments.

In a typical implementation a radio-opaque contrast agent is used so that a position with a polyp will have less contrast agent and will measure a larger back-scattering count. Alternatively, other methods may be used to image the gastrointestinal tract.

U.S. Pat. No. 7,787,926 to Kimchy the disclosure of which is incorporated herein by reference, describes details related to the manufacture and use of such an imaging capsule.

While traversing the gastrointestinal tract, the imaging capsule tends to move erratically in response to pressure forces applied to the imaging capsule. This complicates reconstruction of an image of the path and detecting abnormalities, since the images need to be connected together seamlessly to accurately detect the abnormalities. The erratic motion of the imaging capsule, might cause the imaging capsule to skip a piece of the segment or to sample adjacent segments with a slight bias relative to each other.

It is thus desirable to improve the accuracy in sampling a segment of the gastrointestinal tract, in a way which will enhance the ability to accurately detect abnormalities and seamlessly connect segments.

### SUMMARY OF THE INVENTION

An aspect of an embodiment of the invention, relates to an imaging capsule for scanning inside a living body, including a collimator with two or more output columns for releasing radiation from a radiation source inside the collimator. At least two output column point in a distinct direction and each output column is paired to a detector, so that the imaging capsule can simultaneously scan two or more parallel planes in the vicinity of the imaging capsule to provide measurements of the inner walls of the colon surrounding the imaging capsule. The output column may be designed to scan a complete circumference (e.g. 360°) of the inner walls or part of the circumference (e.g. less than 360°).

The number of output columns may be even or add. Optionally, each output column is directed to scan a distinct segment of a circumference of the surrounding wall around the imaging capsule. Alternatively, some output columns may scan the same plane and the results may be combined or compared to enhance accuracy.

There is thus provided according to an exemplary embodiment of the disclosure, an imaging capsule, comprising:

A radiation source;

A collimator that blocks the emission of radiation from the radiation source except through two or more output columns;

A detector paired to each output column configured to detect particles resulting from X-ray fluorescence and/or Compton backscattering in response to the particles emitted by the output columns;

Wherein the collimator is configured to rotate around an X axis to scan a partial or full inner circumference of a user's colon with radiation emitted from each output column; and wherein at least two of the two or more output columns are tilted by a distinct angle relative to a Y axis that is perpendicular to the X axis, to scan distinct positions along the user's colon and form images of a slice of the colon in parallel planes.

In an exemplary embodiment of the disclosure, two output columns form a straight path from one side of the collimator to an opposite side with the radiation source in the middle of the path. Alternatively or additionally, two output columns tilt by the same absolute angle relative to the Y axis, one tilting toward a forward end of the imaging capsule and one tilting toward a rear end of the imaging capsule. In an exemplary embodiment of the disclosure, each output columns tilts by a different absolute angle relative to the Y axis. Optionally, a pair of output columns form an oblique angle relative to each other. In an exemplary embodiment of the disclosure, the imaging capsule includes three output columns one tilting to a forward end, one tilting to a rear end and one may emit radiation in the direction of the Y axis. Alternatively, the imaging capsule includes three output columns all tilting in the same direction with a different tilt angle. In an exemplary embodiment of the disclosure, the imaging capsule includes an even number of output columns. Optionally, the imaging capsule includes an odd number of output columns. In an exemplary embodiment of the disclosure, at least one output column emits radiation in the direction of the Y axis.

There is further provided according to an exemplary embodiment of the disclosure, a method of imaging with an imaging capsule, comprising:

Providing an imaging capsule, including a radiation source within a collimator that blocks the emission of radiation from the radiation source except through one or more output columns; and a detector paired to each output column configured to detect particles resulting from X-ray fluorescence and/or Compton backscattering in response to the particles emitted by the output columns;

Rotating the collimator around an X axis to scan a partial or full inner circumference of a user's colon with radiation emitted from each output column;

Scanning at least two distinct positions along the user's colon to form images of a slice of the colon in parallel planes with the two or more output columns; wherein at least two output columns are tilted by a distinct angle relative to a Y axis that is perpendicular to the X axis.

### BRIEF DESCRIPTION OF THE DRAWINGS

The present invention will be understood and better appreciated from the following detailed description taken in conjunction with the drawings. Identical structures, elements or parts, which appear in more than one figure, are generally labeled with the same or similar number in all the figures in which they appear, wherein:

FIG. 1 is a schematic illustration of an internal view of an imaging capsule, according to an exemplary embodiment of the invention;

FIG. 2 is a schematic illustration of a cross sectional view of a collimator with two output columns, according to an exemplary embodiment of the disclosure;

FIG. 3A is a schematic illustration of an alternative cross sectional view of a collimator with two output columns, according to an exemplary embodiment of the disclosure;

FIG. 3B is a schematic illustration of a transparent view of an alternative collimator with two output columns, according to an exemplary embodiment of the disclosure; and

FIG. 4 is a schematic illustration on an alternative transparent view of a collimator with three output columns, according to an exemplary embodiment of the disclosure.

### DETAILED DESCRIPTION

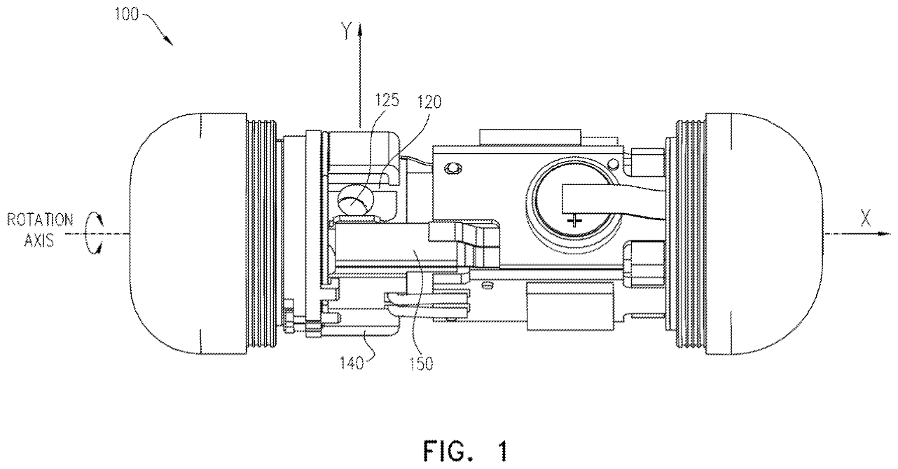

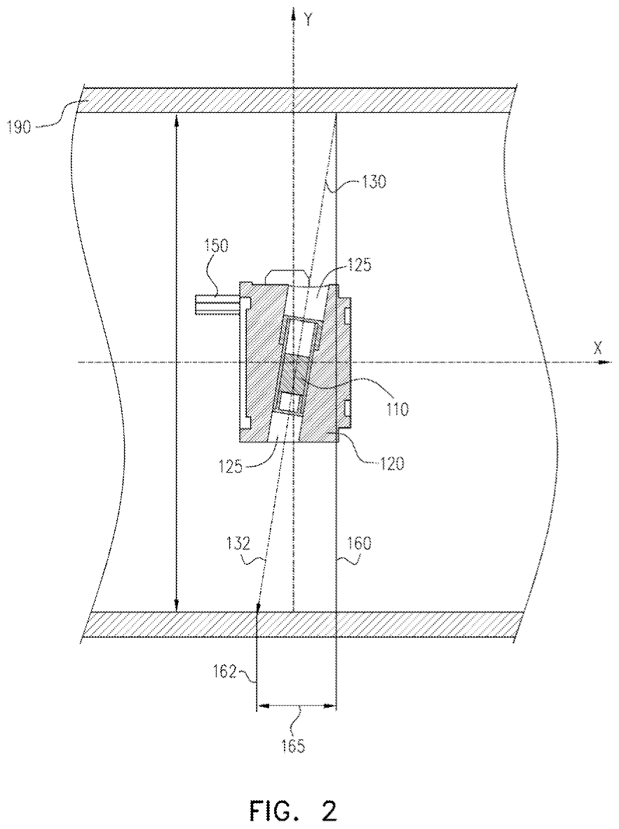

FIG. 1 is a schematic illustration of an internal view of an imaging capsule 100 and FIG. 2 is a cross sectional view of a collimator 120 of the imaging capsule 100 with two output columns 125, according to an exemplary embodiment of the disclosure. In an exemplary embodiment of the invention, a patient swallows a contrast agent which mixes with the content of their gastrointestinal tract to increase the accuracy of radiation measurements. Then the patient swallows imaging capsule 100 to examine the gastrointestinal tract (and especially the colon 190) as the imaging capsule 100 proceeds through the gastrointestinal tract. In an exemplary embodiment of the invention, imaging capsule 100 includes a radiation source 110 (FIG. 2) to provide an X-ray beam. The imaging capsule further comprises collimator 120 with multiple output columns 125 (e.g. at least two as shown in FIG. 2) for directing radiation to scan in different planes. Optionally, each output column 125 is matched with a shutter 140, which can be moved to selectively block the emission of radiation from the radiation source 110 for a specific output column 125. Additionally, each output column 125 may be tilted relative to the collimator 120 and directed to a different position along the colon 190. Each output column 125 is paired with a detector 150 to independently detect X-ray fluorescence and/or Compton backscattering particles in response to the radiation emitted from the output columns 125.

In an exemplary embodiment of the disclosure, the collimator 120 rotates around an axis X, for example along an elongated body of the imaging capsule 100 so that radiation beams (130, 132) emitted from each output column 125 scans a partial or full inner circumference of the user's colon 190 or other organs of the gastrointestinal tract. Optionally, the collimator 120 may be configured to rotate back and forth around the X axis to scan a sector of the inner wall of the colon 190 (e.g. 180° or 270°) with beams (130, 132). In an exemplary embodiment of the disclosure, by scanning simultaneously different positions along the colon 190 with two or more independent beams (130, 132) and monitoring the response for each beam with an independent detector 150, the imaging capsule obtains accurately synchronized images from two or more adjacent positions along the colon 190. As the imaging capsule traverses the colon 190 it will obtain overlapping images that can be analyzed to accurately cover the entire colon 190.

In an exemplary embodiment of the disclosure, the output columns 125 are tilted relative to a Y axis (that is aligned with the collimator 120 and perpendicular to the X axis), for example by about 15° to 25°. Optionally, the tilt angle will be small to enhance accuracy of the detected measurements by not deviating too much from the position of the collimator 120. Alternatively, even large tilt angles may be used, for example 70°-80°. Optionally, the respective detector 150 can be positioned closer to the forward end of the imaging capsule 100 instead of adjacent to the collimator 120, to enhance detection of returned particles when using a large tilt angle for one of the output columns 125.

In an exemplary embodiment of the disclosure, a first beam 130 tilts toward a forward end of the imaging capsule 100 and a second beam 132 tilts in an opposite direction along the X axis, e.g. toward a rear end of the imaging capsule 100. Accordingly, the first beam 130 scans a slice of the colon 190 in a first plane 160 and the second beam 132 scans a slice of the colon 190 in a second plane 162 parallel to the first plane 160, with a distance 165 between the planes that is a function of the tilt angles of the two output columns 125 relative to the Y axis. A benefit of scanning in two (or more) parallel planes simultaneously while the imaging capsule is in a specific position is that abnormalities (e.g. polyps) can be seen more accurately since motion of the imaging capsule 100 does not interfere between the measurements taken for two or more adjacent planes. Likewise multiple planes help to discriminate between polyps, non-polyp structures and gas bubbles. In contrast comparing single planes may lead to errors due to small shifts in the position of the imaging capsule 100 between scanning of adjacent planes, thus causing the imaging capsule 100 to miss small abnormalities.

In an exemplary embodiment of the disclosure, by cross correlating images taken in different positions with images formed from multiple planes scanned by the imaging capsule from a stationary position, the longitudinal distance and longitudinal velocity of the imaging capsule 100 traversing the colon 190 can be determined more accurately.

In some embodiments of the disclosure, a pair of output columns 125 are provided in the form of an essentially straight path from one side of the collimator 120 to another side with the radiation source 110 at the center. Optionally, the output columns 125 are tilted relative to the Y axis by the same absolute angle (one forward and one to the rear of imaging capsule 100). Alternatively the angle on each side of the Y axis may be different for each output column 125.

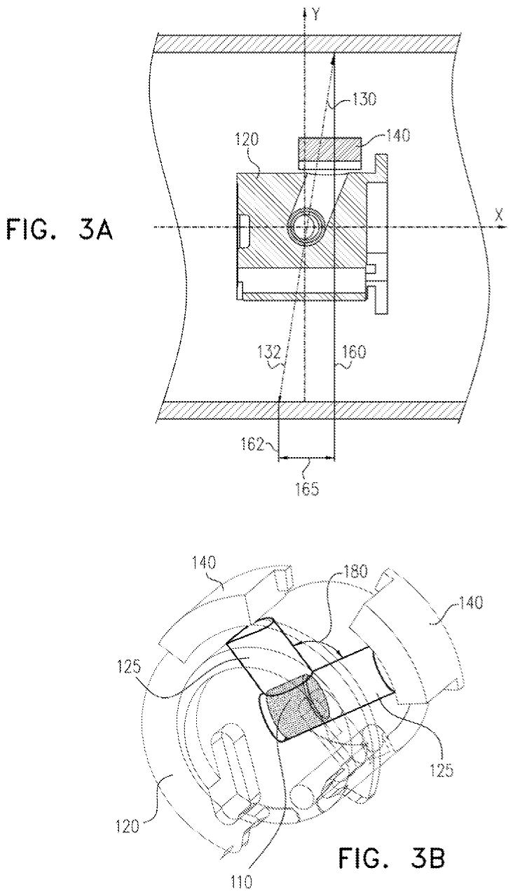

FIG. 3A is a schematic illustration of a cross sectional view of an alternative collimator 120 with two output columns 125 and FIG. 3B is a schematic illustration of a transparent view of alternative collimator 120 with two output columns 125, according to an exemplary embodiment of the disclosure. In some embodiments of the disclosure, the output columns 125 form an oblique angle 180 relative to each other, for example an angle of about 100° relative to each other as shown in FIG. 3A and FIG. 3B. Optionally, such an angle prevents cross talk between the detectors 150 of each of the output columns 125 so that an associated detector ISO does not record particles resulting from the other output column 125. Likewise, the output columns 125 each tilt at a different angle relative to the Y axis to form distinct planes. In some embodiments of the disclosure, a pair of output columns tilt in opposite directions relative to the Y axis. Alternatively, they may tilt in the same direction relative to the Y axis. In some embodiments of the disclosure, one output column may emit radiation in the direction of the Y axis.

In an exemplary embodiment of the disclosure, the number of output columns 125 may be odd or even, for example forming two, three or four independent imaging planes (160, 162) parallel to each other. Optionally, the images in the planes may be full (360°) or partial (less than 360°).

In an exemplary embodiment of the disclosure, when partial planes are scanned, data from consecutive partial sectors can be combined together to form a 3D reconstruction around the entire inner circumference of the colon 190.

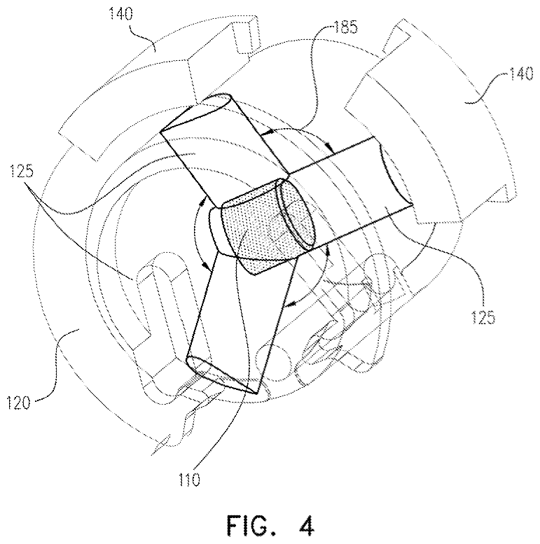

FIG. 4 is a schematic illustration of a transparent view of an alternative collimator 120 with three output columns, according to an exemplary embodiment of the disclosure. In an exemplary embodiment of the disclosure, the three output columns may all tilt in the same direction with different angles or one may tilt to the forward end of imaging capsule 100 (e.g. +15°), one may tilt to the rear end of imaging capsule 100 (e.g. −15°) and one may emit radiation in the direction of the Y axis (e.g. a tilt with an angle of zero). Optionally, the three output columns 125 may be positioned evenly around the Y axis, for example with an angle 185 of 120° between each output column 125. Alternatively, the angle 185 may be different between each two output columns 125. Optionally, the angle 185 is selected to minimize cross talk between the output columns 125 and the detectors 150.

In some embodiments of the disclosure, two output columns 125 may scan the same plane (160, 162) or part of the same plane. Optionally, the results may be combined or compared to enhance accuracy.

It should be appreciated that the above described methods and apparatus may be varied in many ways, including omitting or adding steps, changing the order of steps and the type of devices used. It should be appreciated that different features may be combined in different ways. In particular, not all the features shown above in a particular embodiment are necessary in every embodiment of the invention. Further combinations of the above features are also considered to be within the scope of some embodiments of the invention.

It will be appreciated by persons skilled in the art that the present invention is not limited to what has been particularly shown and described hereinabove. Rather the scope of the present invention is defined only by the claims, which follow.

I/We claim:

1. An imaging capsule, comprising:
a radiation source;
a collimator that blocks the emission of radiation from the radiation source except through two or more output columns;
a detector paired to each output column configured to detect particles resulting from X-ray fluorescence and/or Compton backscattering in response to the particles emitted by the output columns;
wherein the collimator is configured to rotate around an X axis to scan a partial or full inner circumference of a user's colon with radiation emitted from each output column; and wherein at least two of the two or more output columns are tilted by a distinct angle relative to a Y axis that is perpendicular to the X axis, to scan distinct positions along the user's colon and form images of a slice of the colon in parallel planes.

2. The imaging capsule according to claim 1, wherein two output columns form a straight path from one side of the collimator to an opposite side with the radiation source in the middle of the path.

3. The imaging capsule according to claim 1, wherein two output columns tilt by the same absolute angle relative to the Y axis, one tilting toward a forward end of the imaging capsule and one tilting toward a rear end of the imaging capsule.

4. The imaging capsule according to claim 1, wherein each output columns tilts by a different absolute angle relative to the Y axis.

5. The imaging capsule according to claim 1, wherein a pair of output columns form an oblique angle relative to each other.

6. The imaging capsule according to claim 1, wherein the imaging capsule includes three output columns one tilting to a forward end, one tilting to a rear end and one may emit radiation in the direction of the Y axis.

7. The imaging capsule according to claim 1, wherein the imaging capsule includes three output columns all tilting in the same direction with a different tilt angle.

8. The imaging capsule according to claim 1, wherein the imaging capsule includes an even number of output columns.

9. The imaging capsule according to claim 1, wherein the imaging capsule includes an odd number of output columns.

10. The imaging capsule according to claim 1, wherein at least one output column emits radiation in the direction of the Y axis.

11. A method of imaging with an imaging capsule, comprising:
providing an imaging capsule, including a radiation source within a collimator that blocks the emission of radiation from the radiation source except through one or more output columns; and a detector paired to each output column configured to detect particles resulting from X-ray fluorescence and/or Compton backscattering in response to the particles emitted by the output columns;
rotating the collimator around an X axis to scan a partial or full inner circumference of a user's colon with radiation emitted from each output column;
scanning a distinct position along the user's colon to form images of a slice of the colon in parallel planes by having at least two of the two or more output columns, tilted by a distinct angle relative to a Y axis that is perpendicular to the X axis.

12. The method according to claim 11, wherein two output columns form a straight path from one side of the collimator to an opposite side with the radiation source in the middle of the path.

13. The method according to claim 11, wherein two output columns tilt by the same absolute angle relative to the Y axis, one tilting toward a forward end of the imaging capsule and one tilting toward a rear end of the imaging capsule.

14. The method according to claim 11, wherein each output columns tilts by a different absolute angle relative to the Y axis.

15. The method according to claim 11, wherein a pair of output columns form an oblique angle relative to each other.

16. The method according to claim 11, wherein the imaging capsule includes three output columns one tilting to a forward end, one tilting to a rear end and one may emit radiation in the direction of the Y axis.

17. The method according to claim 11, wherein the imaging capsule includes three output columns all tilting in the same direction with a different tilt angle.

18. The method according to claim 11, wherein the imaging capsule includes an even number of output columns.

19. The method according to claim 11, wherein the imaging capsule includes an odd number of output columns.

20. The method according to claim 11, wherein at least one output column emits radiation in the direction of the Y axis.
